(19) United States
(12) Patent Application Publication
(10) Pub. No.: US 2026/0248423 A1
(43) Pub. Date: Aug. 27, 2026

(54) FLEXIBLE BIOSENSOR FOR THE DETECTION AND/OR 2D/3D MAPPING OF BIOMARKER CONCENTRATION

(71) Applicant: Jinghua LI

(72) Inventor: Jinghua LI

(21) Appl. No.: 18/992,248

(22) PCT Filed: Jul. 10, 2023
(86) PCT No.: PCT/US2023/027291

§ 371 Date: Jan. 8, 2025

(51) Int. Cl. A61B 5/1486; A61B 5/1473

(52) U.S. Cl. CPC A61B 5/14865; A61B 5/14735

(57) ABSTRACT

Disclosed are biosensors for detecting an analyte of interest. These biosensors can comprise one or more electrochemical sensors disposed on a flexible substrate. Each of the one or more electrochemical sensors can comprise a first electrode disposed on a flexible substrate; a second electrode disposed on the flexible substrate and in electrochemical contact with the first electrode; and an active layer comprising an enzyme disposed on the first electrode. The biosensor can exploit the fact that many co-substrates or products of the reaction catalyzed by the enzyme are electroactive and/or that reaction of the enzyme with the analyte of interest can involve a redox process that can generate an electronic current in proportion to the concentration of the analyte of interest. These biosensors can thus be used to measure the concentration of an analyte of interest in contact with the active layer by measuring sensor current and/or potential.

### CROSS REFERENCE TO RELATED APPLICATIONS

This application claims the benefit of U.S. Provisional Application No. 63/359,660, filed Jul. 8, 2022, which is incorporated herein by reference in its entirety.

### BACKGROUND

Biofluids contain a diverse range of chemical biomarkers that can provide insights into health and age-related conditions. These biomarkers are highly relevant to fields such as biomedical research, advanced healthcare, and clinical medicine. Continuous monitoring of biomarker levels, in particular, can offer valuable evidence that enables the effective diagnosis and treatment of chronic diseases and injuries. As one example, glutamate is one of the most important excitatory neurotransmitters in the mammalian central nervous system (CNS) that is responsible for learning, memory, and communication between neurons. However, excessive glutamate can lead to hyperexcitability of post synaptic neurons, resulting in induced excitotoxicity. This phenomenon can result in decreased neuronal regeneration and dendritic branching which can not only impair memory and cognition but increase the risk of the development of various neurological diseases. Accordingly, elevated glutamate concentrations show a strong correlation with many neurological disorders, such as traumatic brain injury (TBI), Alzheimer's disease, stroke, epilepsy, chronic pain, and migraines. In patients who have suffered an ischemic stroke and neurological deterioration, glutamate concentrations in the brain can increase from 15 to 200 μM, and in plasma from 100 to 300 μM. Glutamate concentrations that exceed 200 μM in plasma can serve as a predictor of neuronal damage progression at 48 h post stroke. Thus, the development of an implantable glutamate sensor that can relay real-time glutamate concentration levels to medical professionals would aid in both the diagnosis and prevention of stroke in high-risk patients (e.g., smokers, patients with high cholesterol or blood pressure).

However, the accurate, continuous, and real-time measurement of glutamate in situ remains a challenging topic. Conventional techniques, such as glutamate microdialysis and ultraperformance liquid chromatography-tandem mass spectrometry (UPLC-MS/MS), have limited capability for real-time monitoring of subtle changes in glutamate concentration with a spatial resolution. Recently developed glutamate sensor protein, iGluSnFr, enables the optical measurement of glutamate dynamics. However, it only measures glutamate semi-quantitatively without providing an absolute concentration reading and needs genetic modification of the subject. Through the use of electrochemical amperometric techniques, alternative strategies have successfully leveraged biosensors to continuously monitor glutamate dynamics in real-time and/or explore their correlations with electrophysiological signals. Despite the great advances enabled by the pioneering studies, the existing approaches still involve a complex collection of on-chip hardware for signal generation, such as potentiostats, and power supply/management systems. The difficulty in minimizing the form factors of these subsystems poses substantial challenges in achieving the goal of building miniaturized and light weight neural interfaces for understanding and intervening various neurological diseases.

Accordingly, there remains a need for improved sensors that can address these and other needs.

### SUMMARY

Described herein are biosensors for detecting an analyte of interest (e.g., a biomarker of interest). These biosensors can comprise one or more electrochemical sensors disposed on a flexible substrate. Each of the one or more electrochemical sensors can comprise a first electrode disposed on a flexible substrate; a second electrode disposed on the flexible substrate and in electrochemical contact with the first electrode; and an active layer comprising an enzyme disposed on the first electrode. The analyte of interest can be a biochemical substrate for the analyte of interest.

The biosensor can exploit the fact that many co-substrates or products of the reaction catalyzed by the enzyme are electroactive and/or that reaction of the enzyme with the analyte of interest can involve a redox process that can generate an electronic current in proportion to the concentration of the analyte of interest. These biosensors can thus be used to measure the concentration of an analyte of interest (or a co-substrate or product proportional to the concentration of the analyte of interest) in contact with the active layer (e.g., by relation of sensor current and/or potential to the analyte concentration through a suitable calibration).

Representative examples of suitable enzymes may include glucose oxidase, glucose dehydrogenase, NADH oxidase, uricase, urease, creatininase, sarcosine oxidase, creatinase, creatine kinase, creatine amidohydrolase, cholesterol esterase, cholesterol oxidase, glycerol kinase, hexokinase, glycerol-3-phosphate oxidase, lactate oxidase, lactate dehydrogenase, alkaline phosphatase, alanine transaminase, aspartate transaminase, amylase, lipase, esterase, gamma-glutamyl transpeptidase, a glutamate oxidase (e.g., L-glutamate oxidase), pyruvate oxidase, diaphorase, bilirubin oxidase, and mixtures thereof. In certain embodiments, the enzyme can comprise an oxidoreductase, such as a glucose oxidase or a glutamate oxidase.

In some embodiments, the biosensor can comprise a plurality of the electrochemical sensors described above disposed on the flexible substrate. The plurality of the electrochemical sensors can be disposed in a 2-dimensional or 3-dimensional array on the flexible substrate. As such, measurement of the concentration of an analyte of interest at each of the plurality of electrochemical sensors can provide 2-dimensional or 3-dimensional information about the concentration of the analyte of interest at varying points in a medium in contact with the biosensor. The biosensors can be flexible, biocompatible, lightweight, passive, and/or implantable.

For example, provided herein are biosensors for detecting an analyte of interest that comprise one or more electrochemical sensors disposed on a flexible substrate. Each of the one or more electrochemical sensors can comprise: a first electrode disposed on a flexible substrate; a second electrode disposed on the flexible substrate and in electrochemical contact with the first electrode; and an active layer comprising an enzyme disposed on the first electrode. The analyte of interest can be a biochemical substrate for the analyte of interest.

In some embodiments, the active layer can further comprise an electron mediator, such as tetrathiafulvalene.

In some embodiments, the biosensor can further comprise a polymer network disposed on the active layer. The polymer network can be permeable towards the analyte of interest. The polymer network can be configured to immobilize the active layer on the first electrode. In certain embodiments, the polymer network can comprise a sulfonated tetrafluoroethylene-based fluoropolymer-copolymer (e.g., Nafion). In some embodiments, the polymer network can comprise a polymer membrane. In some examples, the membrane can exhibit a 1100 g/mol equivalent weight and/or a thickness of about 0.007 inches. In some embodiments, the polymer network can be substantially impermeable to an interferent.

In some embodiments, the polymer network can be protein modified. For example, in some embodiments, the polymer network can be BSA surface-modified.

In some embodiments, the first electrode comprises a high surface area conductive material, such as Pt black or carbon nanotubes, disposed on a conductive material, such as a metal (e.g., Au).

In some embodiments, the flexible substrate comprises a polymer such as a polyimide. In some embodiments, the enzyme can be chosen from glucose oxidase, glucose dehydrogenase, NADH oxidase, uricase, urease, creatininase, sarcosine oxidase, creatinase, creatine kinase, creatine amidohydrolase, cholesterol esterase, cholesterol oxidase, glycerol kinase, hexokinase, glycerol-3-phosphate oxidase, lactate oxidase, lactate dehydrogenase, alkaline phosphatase, alanine transaminase, aspartate transaminase, amylase, lipase, esterase, gamma-glutamyl transpeptidase, a glutamate oxidase (e.g., L-glutamate oxidase), pyruvate oxidase, diaphorase, bilirubin oxidase, and mixtures thereof.

In certain embodiments, the analyte of interest comprises glutamate and the enzyme comprises glutamate oxidase.

In some embodiments, each of the one or more electrochemical sensors further comprised a third electrode disposed on the flexible substrate and in electrochemical contact with the first electrode and the second electrode.

In some embodiments, the biosensor further comprises circuitry configured to measure a current flowing between the first electrode and the second electrode. In certain embodiments, the biosensor further comprises a processor configured to calculate a concentration of the analyte of interest in contact with the first electrode based on the current flowing between the first electrode and the second electrode.

In some embodiments, the biosensor further comprises circuitry configured to measure a potential between the first electrode and the second electrode. In certain embodiments, the biosensor further comprises a processor configured to calculate a concentration of the analyte of interest in contact with the first electrode based on the potential between the first electrode and the second electrode.

In some embodiments, the biosensor comprises a plurality of the electrochemical sensors disposed on the flexible substrate. If desired, the plurality of the electrochemical sensors can be disposed in a 2-dimensional or 3-dimensional array on the flexible substrate.

In some of these embodiments, the biosensor further comprises circuitry configured to measure a current flowing between the first electrode and the second electrode in each of the plurality of the electrochemical sensors. In some of these embodiments, the biosensor can further comprise a processor configured to calculate a concentration of the analyte of interest in contact with the first electrode in each of the plurality of the electrochemical sensors based on the current flowing between the first electrode and the second electrode. In certain embodiments, the processor can further be configured to correlate the concentration of the analyte of interest in contact with the first electrode of each of the plurality of the electrochemical sensors with a 2-dimensional or 3-dimensional location of each of the plurality of the electrochemical sensors within the 2-dimensional or 3-dimensional array, thereby providing 2-dimensional or 3-dimensional information regarding the concentration of the analyte of interest in proximity to the biosensor.

In other of these embodiments, the biosensor further comprises circuitry configured to measure a potential between the first electrode and the second electrode in each of the plurality of the electrochemical sensors. In some of these embodiments, the biosensor can further comprise a processor configured to calculate a concentration of the analyte of interest in contact with the first electrode in each of the plurality of the electrochemical sensors based on the potential between the first electrode and the second electrode. In certain embodiments, the processor can further be configured to correlate the concentration of the analyte of interest in contact with the first electrode of each of the plurality of the electrochemical sensors with a 2-dimensional or 3-dimensional location of each of the plurality of the electrochemical sensors within the 2-dimensional or 3-dimensional array, thereby providing 2-dimensional or 3-dimensional information regarding the concentration of the analyte of interest in proximity to the biosensor.

In some embodiments, the biosensor is battery-free. In certain embodiments, the biosensor is not electrically connected to a power source.

In some embodiments, the sensors described herein can be biocompatible, implantable, or any combination thereof. These sensors can be utilized in a wide variety of sensing applications, including biosensing applications.

Also provided are methods of detecting an analyte of interest in a medium that comprise contacting the medium with a sensor described herein. In some examples, the medium can comprise a biological sample, such as bodily fluid or tissue (e.g., in vivo or ex vivo).

### BRIEF DESCRIPTION OF THE FIGURES

FIG. 1. Conceptual illustration of monitoring glutamate release during synaptic transmission in extracellular space using the flexible, miniaturized glutamate sensors and the working principle of the sensing platform inspired by the design and structure of biofuel cells.

FIG. 2. Structures of the functional interface materials in the anode and cathode. (A) Schematic illustration showing the functionalization protocol of active components onto the surfaces of the anode and cathode. (B) SEM image of CNT before any functionalization. (C) SEM image of an electrode with Pt-black electrodeposited at the surface. (D) SEM image of an anode containing CNT after coating with TTF. (E) SEM image of an anode containing CNT after coating with BSA and GlutOx. (F) SEM image of a cathode containing CNT after coating with Pt—C particles. (G) SEM image of a cathode containing CNT after coating with Pt—C particles and Nafion.

FIG. 3. Performance characterization of the glutamate biofuel cells. (A) Change in potential (calibrated response) as a function of time when the glutamate concentration increases by 0.05 mM every 200 s. (B) Magnified view of (A) from 0 to 2000 s, where the arrows indicate the point of glutamate addition. (C) Calibration curve of glutamate biofuel cell with a sensitivity of 5.1721 mV mM⁻¹. (D) Reversibility test whereby 0.25 mM of Glutamate is added into the glutamate biofuel cell. After 3 additions, the biofuel cell is placed in fresh 1X PBS. The data during the three sensing cycles are collected separately with a pause between each cycle and plotted together for comparison. (E) Cyclic voltammetry performed from 0.2 mM to 0.5 mM glutamate in 0.05 mM steps from −0.2 V to 1.0 V with a scanning rate of 0.1 V s⁻¹. (F) Stability test showing response of a glutamate biofuel cell to 1 mM of Glutamate for a period of 46 days. (G) Decay in relative response (%) as time progresses. (H) Response of the glutamate biofuel cell to a glutamate concentration similar to that found in CSF (4.73 μM).

FIG. 4. Impact of key parameters for optimization of the biofuel cell performance. (A) Schematic illustration showing key parameters explored in this study impacting the performance of the biofuel cell glutamate sensors. (B) Effect of different TTF concentrations on sensitivity of the glutamate biofuel cell. (C) Effect of temperature on sensitivity of the glutamate biofuel cell: body temperature (37° C.) and room temperature (25° C.). The red curve is the same as in FIG. 3A for comparison. (D) Change in potential vs. time for a glutamate biofuel cell with 5× enzyme amount of that used in FIG. 3A. (E) Effect of aerating the solution for 5 minutes before characterization on the biofuel cell performance showing the importance of O₂ for eliminating the initial signal drift. (F) Performance of a biofuel cell in the presence of interferents such as dopamine, serotonin (5-HT) and glucose at physiologically relevant concentrations. (G) Schematic illustration of the differential amplifier utilized to amplify signal for miniaturized electrodes. (H) Effect of reducing anode diameter to 1 mm, 0.75 mm and 0.35 mm while the cathode diameter is kept constant at 2.5 mm. (I) Effect of reducing cathode diameter to 1 mm, 0.75 mm and 0.35 mm while the anode diameter is kept constant at 2.5 mm.

FIG. 5. Performance of the glutamate biofuel cell in aCSF and brain tissue homogenate dilutions. (A) Schematic illustration of the fabrication procedure for the biofuel cell-inspired flexible glutamate probe. (B) Schematic illustration of collecting brain homogenate for experimentation with the glutamate assay and microfabricated probe. (C) Photograph and microscopic image of the brain homogenate and glutamate probe used for the study. (D) Change in measured potential of the glutamate probe in aCSF with a stepwise increase in glutamate concentration (0.2 mM). (E) Change in measured potential of the glutamate probe in 50× dilution of brain homogenate with a stepwise increase in glutamate concentration (0.2 mM). (F) Calibration curves of glutamate probe in 50× dilution brain homogenate and aCSF. (G) Pearson's R coefficient for the calculated glutamate concentration vs. the actual glutamate concentration for the 50× dilution brain homogenate. (H) Comparison of calculated glutamate concentrations between the glutamate assay and the glutamate probe.

FIG. 6. Ex vivo study of dynamic glutamate release from the hippocampal circuit in mice using miniaturized glutamate probes. (A) Microscope image of a 50×50 μm² probe. (B) Photograph of a probe on a fingertip. (C) Photograph of a probe penetrating into a 0.6% (w/w) agarose brain model. (D) Change in electric potential measured by the 50×50 μm² glutamate probe in aCSF with a stepwise increase in glutamate concentration (0.2 mM). Inset: extracted calibration plot. (E) Schematic illustration of the hippocampal circuit in mouse and the stimulation/sensing protocols used in this study. (F) Schematic illustration showing the glutamate release and uptake process. This image is created using Biorender.com. (G) Photograph showing the experimental setup for the ex vivo study using a glutamate probe and a stimulation electrode on a mouse brain slice (thickness: 300 μm). (H) Microscopic image of the glutamate probe and the stimulation electrode on the brain slice. The distance between the stimulation electrode and the probe is approximately 400 μm. The cathode of the glutamate probe is in the surrounding aCSF but not in direct contact with the hippocampus. (I) Change in glutamate concentration detected using a miniaturized probe following electrical stimulation with different pulse intensities (2 mA and 0.5 mA), and comparison with results obtained using an unfunctionalized probe (pulse width: 0.05 ms). (J) Change in glutamate concentration with five repeated pulses (intensity: 0.5 mA, width: 0.5 ms) measured using a miniaturized probe. (K) Change in glutamate concentration with three repeated pulses (intensity: 0.5 mA, width: 0.5 mA) measured using a miniaturized probe. (L) Change in glutamate concentration with two repeated pulses (intensity: 2 mA, width: 0.05 mA) measured using a miniaturized probe showing the dynamic increase and decrease process.

FIG. 7. Schematic illustration of the use of the sensors described herein for the real-time monitoring of glutamate dynamics.

FIG. 8. Schematic illustration of a sensor described herein.

### DETAILED DESCRIPTION

### Definitions

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

The term “about” as used herein is a broad term, and is to be given its ordinary and customary meaning to a person of ordinary skill in the art (and is not be limited to a special or customized meaning), and refers without limitation to allowing for a degree of variability in a value or range, for example, within 10%, within 5%, or within 1% of a stated value or of a stated limit of a range, and includes the exact stated value or range.

The term “substantially” as used herein refers to a majority of, or mostly, as in at least about 50%, 60%, 70%, 80%, 90%, 95%, 96%, 97%, 98%, 99%, 99.5%, 99.9%, 99.99%, or at least about 99.999% or more, or 100%. The phrase “substantially free of” as used herein can mean having none or having a trivial amount of, such that the amount of material present does not affect the material properties of the composition including the material, such that about 0 wt % to about 5 wt % of the composition is the material, or about 0 wt % to about 1 wt %, or about 5 wt % or less, or less than or equal to about 4.5 wt %, 4, 3.5, 3, 2.5, 2, 1.5, 1, 0.9, 0.8, 0.7, 0.6, 0.5, 0.4, 0.3, 0.2, 0.1, 0.01, or about 0.001 wt % or less, or about 0 wt %.

As used herein, the term “flexible” itself or when used to modify or describe the sensor and/or components thereof means capable of elastically bending or twisting under loads generated by body movements of the wearer of the sensor when generally in conformal contact with the wearer without disrupting sensor performance.

The terms “implanted” or “implantable” as used herein are broad terms, and are to be given their ordinary and customary meaning to a person of ordinary skill in the art (and are not to be limited to a special or customized meaning), and refer without limitation to objects (e.g., sensors) that are inserted subcutaneously (i.e., in the layer of fat between the skin and the muscle) or transcutaneously (i.e., penetrating, entering, or passing through intact skin), which may result in a sensor that has an in vivo portion and an ex vivo portion.

The term “in vivo” as used herein is a broad term, and is to be given its ordinary and customary meaning to a person of ordinary skill in the art (and is not to be limited to a special or customized meaning), and without limitation is inclusive of the portion of a device (for example, a sensor) adapted for insertion into and/or existence within a living body of a host.

The term “ex vivo” as used herein is a broad term, and is to be given its ordinary and customary meaning to a person of ordinary skill in the art (and is not to be limited to a special or customized meaning), and without limitation is inclusive of a portion of a device (for example, a sensor) adapted to remain and/or exist outside of a living body of a host.

Described herein are biosensors for detecting an analyte of interest (e.g., a biomarker of interest). These biosensors can comprise one or more electrochemical sensors disposed on a flexible substrate. Each of the one or more electrochemical sensors can comprise a first electrode disposed on a flexible substrate; a second electrode disposed on the flexible substrate and in electrochemical contact with the first electrode; and an active layer comprising an enzyme disposed on the first electrode. The analyte of interest can be a biochemical substrate for the analyte of interest.

The biosensor can exploit the fact that many co-substrates or products of the reaction catalyzed by the enzyme are electroactive and/or that reaction of the enzyme with the analyte of interest can involve a redox process that can generate an electronic current in proportion to the concentration of the analyte of interest. These biosensors can thus be used to measure the concentration of an analyte of interest (or a co-substrate or product proportional to the concentration of the analyte of interest) in contact with the active layer (e.g., by relation of sensor current and/or potential to the analyte concentration through a suitable calibration

Representative examples of suitable enzymes may include glucose oxidase, glucose dehydrogenase, NADH oxidase, uricase, urease, creatininase, sarcosine oxidase, creatinase, creatine kinase, creatine amidohydrolase, cholesterol esterase, cholesterol oxidase, glycerol kinase, hexokinase, glycerol-3-phosphate oxidase, lactate oxidase, lactate dehydrogenase, alkaline phosphatase, alanine transaminase, aspartate transaminase, amylase, lipase, esterase, gamma-glutamyl transpeptidase, a glutamate oxidase (e.g., L-glutamate oxidase), pyruvate oxidase, diaphorase, bilirubin oxidase, and mixtures thereof. In certain embodiments, the enzyme can comprise an oxidoreductase, such as a glucose oxidase or a glutamate oxidase.

In some embodiments, the biosensor can comprise a plurality of the electrochemical sensors described above disposed on the flexible substrate. The plurality of the electrochemical sensors can be disposed in a 2-dimensional or 3-dimensional array on the flexible substrate. As such, measurement of the concentration of an analyte of interest at each of the plurality of electrochemical sensors can provide 2-dimensional or 3-dimensional information about the concentration of the analyte of interest at varying points in a medium in contact with the biosensor. The biosensors can be flexible, biocompatible, lightweight, passive, and/or implantable.

For example, referring now to FIG. 8, provided herein are biosensors (100) for detecting an analyte of interest that comprise one or more electrochemical sensors (102) disposed on a flexible substrate (104). Each of the one or more electrochemical sensors can comprise: a first electrode (an anode, 106) disposed on a flexible substrate; a second electrode (a cathode, 108) disposed on the flexible substrate and in electrochemical contact with the first electrode; and an active layer (110) comprising an enzyme (112) disposed on the first electrode. The analyte of interest can be a biochemical substrate for the analyte of interest.

In some embodiments, the active layer (110) can further comprise an electron mediator (114), such as tetrathiafulvalene. Examples of other suitable electron mediators include, for example, Prussian blue, ferrocenecarboxylic acid, ferrocene methanol, benzoquinone, anthraquinone, osmium bipyridyl, methylviologen, and benzylviologen.

In some embodiments, the biosensor can further comprise a polymer network (116) disposed on the active layer. The polymer network can be permeable towards the analyte of interest. In some embodiments, the polymer network can be substantially impermeable to an interferent. The polymer network can be configured to immobilize the active layer on the first electrode.

In certain embodiments, the polymer network can comprise a sulfonated tetrafluoroethylene-based fluoropolymer-copolymer (e.g., Nafion). Other suitable polymer networks include, for example, polyvinyl alcohol (PVA), polyethylene oxide (PEO), polyethylene glycol (PEG), sulfonated polyether ether ketone (SPEEK), chitosan-based membranes, cellulose-based membranes (e.g., regenerated cellulose, cellulose acetate), composite membranes, metal-organic frameworks (MOFs), and covalent organic frameworks (COFs). In some embodiments, the polymer network can comprise a polymer membrane. In some examples, the membrane can exhibit a 1100 g/mol equivalent weight and/or a thickness of about 0.007 inches.

In some embodiments, the polymer network can be protein-modified. For example, in some embodiments, the polymer network can be BSA surface-modified. The surface modification can function to passivate the surface (e.g., to reduce immune response to the sensor surface upon implantation). The surface modification can also serve to improve selectivity of the sensor towards an analyte of interest.

In some embodiments, the first electrode (106) comprises a high surface area conductive material (118), such as Pt black or carbon nanotubes, disposed on a conductive material (120), such as a metal (e.g., Au).

In some embodiments, the flexible substrate comprises a polymer such as a polyimide.

In some embodiments, the enzyme can be chosen from glucose oxidase, glucose dehydrogenase, NADH oxidase, uricase, urease, creatininase, sarcosine oxidase, creatinase, creatine kinase, creatine amidohydrolase, cholesterol esterase, cholesterol oxidase, glycerol kinase, hexokinase, glycerol-3-phosphate oxidase, lactate oxidase, lactate dehydrogenase, alkaline phosphatase, alanine transaminase, aspartate transaminase, amylase, lipase, esterase, gamma-glutamyl transpeptidase, a glutamate oxidase (e.g., L-glutamate oxidase), pyruvate oxidase, diaphorase, bilirubin oxidase, and mixtures thereof.

In certain embodiments, the analyte of interest comprises glutamate and the enzyme comprises glutamate oxidase.

Referring again to FIG. 8, in some embodiments, an electrocatalyst layer (126), such as a catalyst for the reduction of oxygen to water (e.g., a platinized carbon layer), can be disposed on the second electrode (108). In some embodiments, the second electrode can comprise a high surface area conductive material (122), such as Pt black or carbon nanotubes, disposed on a conductive material (124), such as a metal (e.g., Au).

In some embodiments, a polymer network (128) can be disposed on electrocatalyst layer (126). The polymer network can be configured to immobilize the electrocatalyst layer on the second electrode. In certain embodiments, the polymer network can comprise a sulfonated tetrafluoroethylene-based fluoropolymer-copolymer (e.g., Nafion). In some embodiments, the polymer network can comprise a polymer membrane. In some examples, the membrane can exhibit a 1100 g/mol equivalent weight and/or a thickness of about 0.007 inches.

In some embodiments, the polymer network can be protein-modified. For example, in some embodiments, the polymer network can be BSA surface-modified. The surface modification can function to passivate the surface (e.g., to reduce immune response to the sensor surface upon implantation).

In some embodiments, each of the one or more electrochemical sensors further comprised a third electrode disposed on the flexible substrate and in electrochemical contact with the first electrode and the second electrode.

In some embodiments, the biosensor further comprises circuitry configured to measure a current flowing between the first electrode and the second electrode. In certain embodiments, the biosensor further comprises a processor configured to calculate a concentration of the analyte of interest in contact with the first electrode based on the current flowing between the first electrode and the second electrode.

In some embodiments, the biosensor further comprises circuitry configured to measure a potential between the first electrode and the second electrode. In certain embodiments, the biosensor further comprises a processor configured to calculate a concentration of the analyte of interest in contact with the first electrode based on the potential between the first electrode and the second electrode.

In some embodiments, the biosensor comprises a plurality of the electrochemical sensors disposed on the flexible substrate. If desired, the plurality of the electrochemical sensors can be disposed in a 2-dimensional or 3-dimensional array on the flexible substrate.

In some of these embodiments, the biosensor further comprises circuitry configured to measure a current flowing between the first electrode and the second electrode in each of the plurality of the electrochemical sensors. In some of these embodiments, the biosensor can further comprise a processor configured to calculate a concentration of the analyte of interest in contact with the first electrode in each of the plurality of the electrochemical sensors based on the current flowing between the first electrode and the second electrode. In certain embodiments, the processor can further be configured to correlate the concentration of the analyte of interest in contact with the first electrode of each of the plurality of the electrochemical sensors with a 2-dimensional or 3-dimensional location of each of the plurality of the electrochemical sensors within the 2-dimensional or 3-dimensional array, thereby providing 2-dimensional or 3-dimensional information regarding the concentration of the analyte of interest in proximity to the biosensor.

In other of these embodiments, the biosensor further comprises circuitry configured to measure a potential between the first electrode and the second electrode in each of the plurality of the electrochemical sensors. In some of these embodiments, the biosensor can further comprise a processor configured to calculate a concentration of the analyte of interest in contact with the first electrode in each of the plurality of the electrochemical sensors based on the potential between the first electrode and the second electrode. In certain embodiments, the processor can further be configured to correlate the concentration of the analyte of interest in contact with the first electrode of each of the plurality of the electrochemical sensors with a 2-dimensional or 3-dimensional location of each of the plurality of the electrochemical sensors within the 2-dimensional or 3-dimensional array, thereby providing 2-dimensional or 3-dimensional information regarding the concentration of the analyte of interest in proximity to the biosensor.

In some embodiments, the biosensor is battery-free. In certain embodiments, the biosensor is not electrically connected to a power source.

### EXAMPLES

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

### Example 1. Flexible, Miniaturized Sensing Probes Inspired by Biofuel Cells for Monitoring Synaptically Released Glutamate in the Mouse Brain

### Summary

Chemical biomarkers in the central nervous system can provide valuable quantitative measures to gain insight into the etiology and pathogenesis of neurological diseases. Glutamate, one of the most important excitatory neurotransmitters in the brain, has been found to be upregulated in various neurological disorders, such as traumatic brain injury, Alzheimer's disease, stroke, epilepsy, chronic pain, and migraines. However, quantitatively monitoring glutamate dynamics in situ has been challenging. This work presents a novel class of flexible, miniaturized probes inspired by biofuel cells for monitoring synaptically released glutamate in the nervous system. The resulting sensors, with dimensions as low as 50 by 50 μm², can detect real-time changes in glutamate within the biologically relevant concentration range. Experiments exploiting the hippocampal circuit in mice models demonstrate the capability of the sensors in monitoring glutamate release via electrical stimulation using acute brain slices. These advances could aid in basic neuroscience studies and translational engineering, as the sensors provide a diagnostic tool for neurological disorders. Additionally, adapting the biofuel cell design to other neurotransmitters can potentially enable the detailed study of the effect of neurotransmitter dysregulation on neuronal cell signaling pathways and revolutionize neuroscience.

### Introduction

In this Example, we present a class of flexible, miniaturized probes inspired by the structure and working principle of biofuel cells for monitoring synaptically released glutamate in the nervous system. The sensor exploits an enzyme functionalized sensing interface, where the anodic and cathodic reactions will spontaneously generate electrical currents proportional to the concentration of glutamate in solution. The elimination of potentiostat simplifies the measurement setup compared to that for conventional three-electrode electrochemical cells. Systematic studies investigate the structure-performance interrelationship of the enzyme-functionalized interface for quantitative analysis. The resulting sensors, with dimensions as small as 50 by 50 μm², can detect real-time changes in glutamate within the biologically relevant concentration range (μM level in cerebrospinal fluids (CSF), μM-mM level near synaptic vesicle release sites). Ex vivo experiments using the hippocampal circuit in mice models demonstrate the capability of the sensors in monitoring glutamate release during synaptic transmission in electrically stimulated acute brain slices. By functioning as sensing media for brain-machine interfaces, the resulting devices have the potential to improve our understanding of the underlying molecular and cellular mechanisms of structural and functional maladaptive changes in neural circuits after a variety of CNS trauma and diseases. This knowledge can shed light on the development of new therapeutic interventions for these pathological conditions.

### Results and Discussion

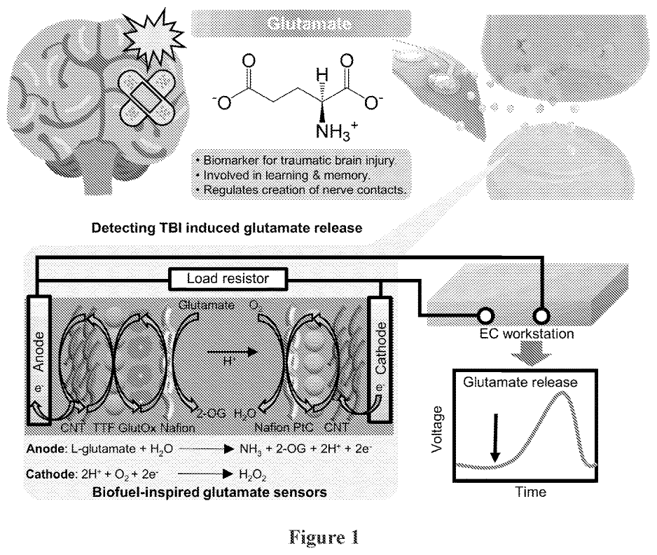

Working principle and fabrication procedures of the glutamate biofuel cell sensors. Inspired by biofuel cell structures, we designed the glutamate sensors developed in this Example, where glutamate spontaneously generates electrical signals proportional to the concentration. FIG. 1 illustrates the primary components and the working principle of the sensing platform. The anode included a gold layer, a rough electrically conductive layer to increase the surface area (e.g., platinum black (Pt-black) or carbon nanotubes (CNTs)), a redox mediator layer containing tetrathiafulvalene (TTF) for electron transfer, an enzyme layer containing immobilized glutamate oxidase (GlutOx) linked to bovine serum albumin (BSA), and a protective layer of Nafion against interferent molecules. The rough, electrically conductive surface improved the transfer of electrons from the redox mediators and enzyme layers to the gold electrode, and increased the surface area for the attachment of enzymes. The redox mediator TTF aided in the transfer of electrons from glutamate that has reacted with GlutOx to the electrode. The enzyme layer contained GlutOx as the catalyst and BSA conjugated to GlutOx to minimize leaching to the surrounding solution. The reaction mechanism for the oxidation of glutamate is as follows:

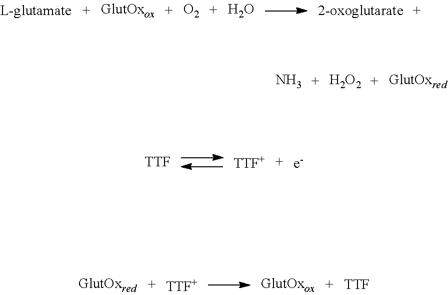

Glutamate undergoes oxidation to produce 2-oxoglutarate (2-OG), ammonia and hydrogen peroxide which simultaneously converts glutamate oxidase into a reduced form. TTF goes through a reversible reaction in which it exists in chemical equilibrium between TTF and TTF⁺ once immersed in solution. TTF⁺ reacts with the reduced form of GlutOx, and the reaction converts the enzyme and TTF back to their original states.

The cathode is composed of a gold layer, a rough electrically conductive surface layer of either Pt-black or CNTs, a platinized carbon layer (Pt—C) layer, and a layer of Nafion. The functions of the Nafion and Pt-black/CNT are similar to those in the anode, and the Pt—C layer acts as a catalyst for the reduction of oxygen to water. The anodic and cathodic reactions generate electrical currents proportional to the concentration of the glutamate. A load resistor connecting the anode and cathode transforms the current into a voltage for signal readout using an electrochemical workstation.

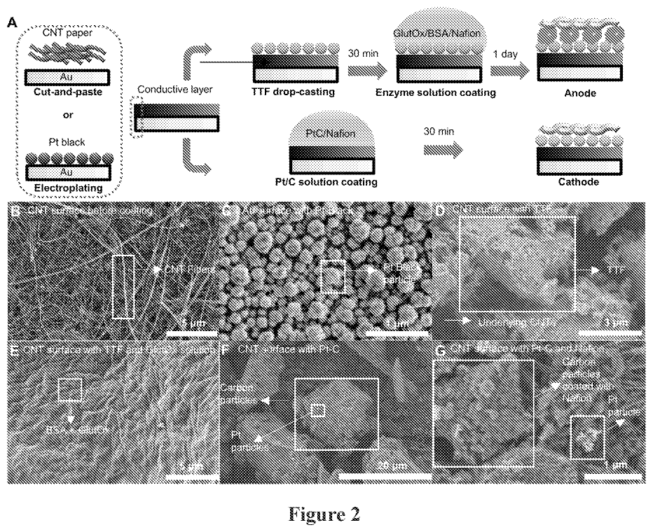

FIG. 2(A) illustrates the procedures for the preparation and functionalization of the electrodes. After the preparation of the conductive layer on the gold electrode, drop-casting TTF yields a thin redox mediator layer on the surface. Then, drop-casting a mixture of BSA, Nafion and GlutOx followed by drying the system at 4° C. for a day forms the enzyme coating layer. To prepare the cathode, drop-casting a solution of Pt—C and Nafion deposits the catalyst for the reduction of oxygen.

FIG. 2B-G display scanning electron microscope (SEM) images illustrating the changes in surface morphology of the electrodes during the fabrication process. FIG. 2(B) exhibits the fibrous structure of the CNT surface before any modifications. FIG. 2(C) highlights the structure of an Au surface after the electrodeposition of Pt-black as an alternative conductive layer. FIG. 2(D) shows large TTF agglomerates interspersed within the CNT network. FIG. 2(E) displays the surface of CNT after the addition of the enzyme layer, which covers both the underlying CNT network and the TTF agglomerates, leading to a smooth surface morphology. FIG. 2(F) shows Pt—C drop casted onto the CNTs, with small Pt particles on the surface of the larger carbon particles. FIG. 2(G) presents the surface of CNT with both Pt—C and Nafion deposited, where the surface of the Pt—C particles appears rougher due to the addition of Nafion.

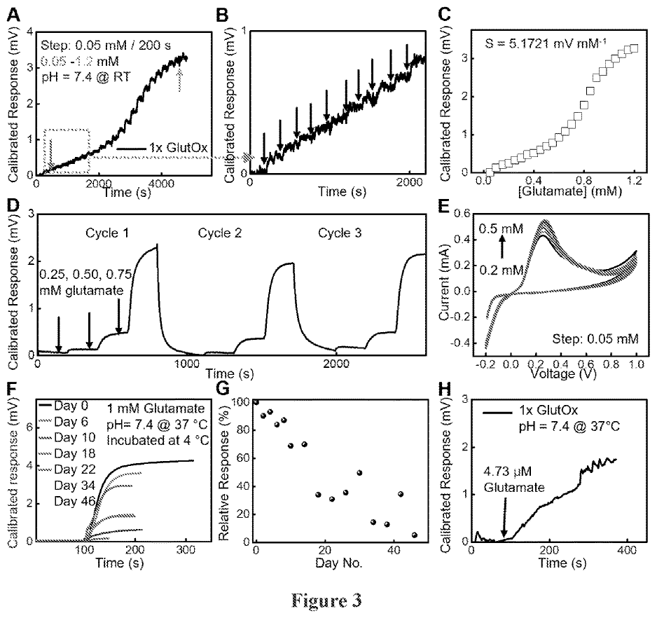

Sensing performance characterization of the glutamate sensors. The glutamate biofuel cell sensors operate by measuring the voltage across the load resistor that connects the anode and cathode. This voltage corresponds to the glutamate concentration, with higher concentrations yielding higher signal readouts. First, we assess the performance of the sensors in solutions with the known concentration of glutamate (FIG. 3). Testing of the sensor with a pair of commercial Au disk electrodes (diameter: 2.5 mm) takes place by periodically adding 50 μM glutamate every 200 s in 1× phosphate-buffered saline (PBS) while recording the stepwise change in potential (FIG. 3(A)-(C)) (load resistor: 100 kΩ). The sensor has a working range of 0.05 mM to 1.2 mM, with a linear response in the range of 0.6 mM to 1.0 mM. Extracting the slope of the curve within the linear range of detection yields a sensitivity of 5.1721 m V mM⁻¹ Calculating the standard deviation of the baseline of equilibrated signals (in this case, the last 100 s before the addition of glutamate) estimates the noise level (0.012 mV). The limit of detection (LOD), which is the lowest concentration of an analyte in a sample that can be detected with a stated probability, is 0.0069 mM, as estimated using the equation 3× (the noise of the system/the sensitivity of the system).

The trend observed is that, at lower glutamate concentrations (0.05 mM to 0.6 mM), there is a slight increase in potential because the current generated from the enzymatic reaction is minimal, as glutamate only binds to a small number of active sites of GlutOx. Similarly, at higher concentrations (1.0 mM to 1.2 mM), the response in potential is lower due to the surface saturation. Such biofuel cell sensors have the potential to encompass physiologically relevant concentration ranges of glutamate in the CNS system as previous studies have reported the concentration of glutamate in the synaptic cleft to be 1 mM, while the ambient concentration in CSF ranges from ~2.54 to 6.51 μM.

To investigate the reversibility of the glutamate sensors, we increase glutamate concentration in a stepwise fashion (250 μM) and then reduce it to 0 afterward for three consecutive cycles (FIG. 3(D)). The results indicate that the sensing platform has a sensitivity of approximately 2.77 mV mM⁻¹ throughout the three cycles and returns to a similar baseline after being placed back into fresh PBS solution. FIG. 3(E) shows cyclic voltammetry scans of the anode with increasing glutamate concentration added in 1×PBS. The amplitude of the oxidation peak at 0.35V becomes larger as the glutamate concentration increases from 0.2 to 0.5 mM. This oxidation peak corresponds to that of TTF, consistent with previous reports in the literature, further confirming the function of TTF in electron shuttling.

Store freshly prepared sensors in 1×PBS at 4° C. retains the bioactivity of enzymes. FIGS. 3(F) and 3(G) show the lifetime of such sensors in a liquid environment. The results suggest that the sensor can maintain 90%, 80%, 70% of its original sensitivity on Day 0 for 4, 8, and 14 days, respectively. Eventually, the system loses 95% of its sensitivity on Day 46, possibly due to the leaching of functional components into the solution.

FIG. 3(H) showcases the performance of the sensor in artificial CSF (aCSF) at body temperature (37° C.). Increasing the amount of glutamate to 4.73 μM (which is at the same order of magnitude as the concentration found in ambient CSF according to the literature) induces an increase in potential. Overall, the performance characterization presented here suggests the feasibility of using the biofuel cell sensor for detecting glutamate at physiologically relevant concentrations.

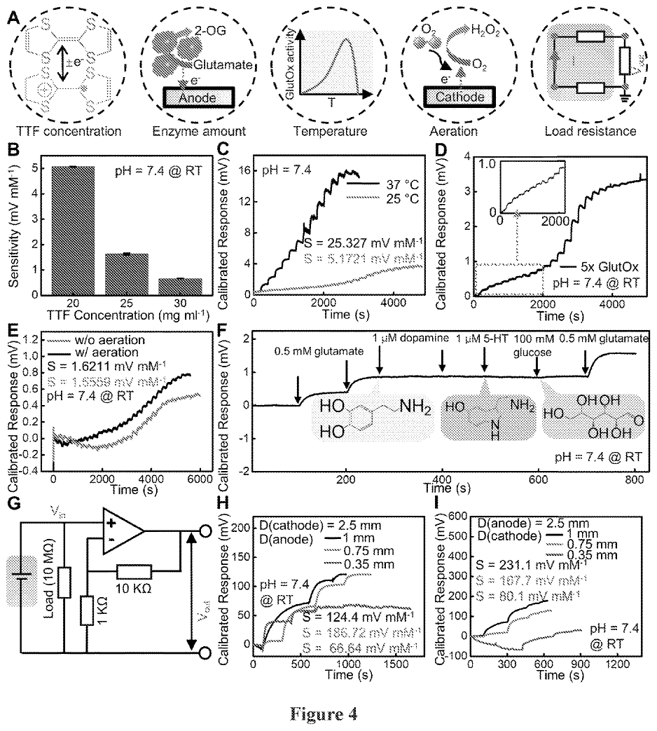

Structure-performance interrelationship of biofuel cell sensors. Systematic studies investigate key parameters impacting the performance of the biofuel cell sensors with a summary presented in FIG. 4(A). FIG. 4(B) demonstrates the impact of TTF concentration on sensitivity, with results indicating that increasing the TTF concentration leads to a decrease in sensitivity (a decrease of 5.1721 mV mM⁻¹ at 20 mg ml⁻¹ to 0.6676 m V mM⁻¹ at 30 mg ml⁻¹). One possible explanation is that a lower concentration of TTF may lead to the crystallization of TTF particles at a smaller size, maximizing the surface area available for catalyzing the reaction. FIG. 4(C) illustrates the difference in sensitivity between at body temperature (25.327 mV mM⁻¹ at 37° C.) and room temperature (5.1721 mV mM⁻¹ at 25° C.) in 1×PBS. The extracted value is ~5 times higher at the higher temperature due to both the increased diffusion rate of the substrate and the elevated activity of the enzyme. FIG. 4(D) shows the effect of adding 5 times the amount of enzyme solution to the surface of the electrode. While the sensitivity is only slightly higher (5.17 mV mM⁻¹ for the original vs. 5.74 mV mM⁻¹ for the 5× amount), the more significant benefit is the reduced noise level (0.012 mV for the original vs. 0.00476 mV for the 5× amount), which is attributed to both the enhanced charge transfer and additional amount of Nafion mixed with the enzyme solution as the surface passivation layer. The effect of aeration of the test solution on sensor performance appears in FIG. 4(E). The sensor response shows drift without aeration, while no such decrease in potential presents for sensors with aeration. The initial decrease in potential may be due to voltage reversal, which can occur when there is insufficient substrate (i.e., glutamate) or insufficient oxygen (i.e., the “oxygen starvation” effect) for the cathodic reaction to occur, resulting in the undesired switch in the polarity of the sensors.

FIG. 4(F) illustrates the capability of Nafion in protecting the fuel cell from interfering molecules such dopamine, serotonin and glucose with physiologically relevant concentrations to ensure a high selectivity. Previous studies have reported the physiological concentrations of dopamine and serotonin for dopaminergic and serotonergic neurons during an action potential are 0.25 UM and 0.1 μM, respectively, which justify the selection of concentrations of interferents used here. The sensor exhibits no significant change in potential in the presence of interferents when compared to the change in potential from glutamate (**** p<0.0001).

To ensure minimal invasiveness of the neural interface to surrounding bio-tissues in animal models, the design of miniaturized electrode pairs is highly desirable. Pt-black provides a realistic option for creating the biochemical interface on miniaturized electrodes through electrodeposition. We evaluated the performance utilizing electrodeposited Pt-black nanoparticles on a commercial gold electrode as a replacement for CNTs on the biochemical interface with the functionalization protocol kept constant. Initially, a voltage decrease occurs due to voltage reversal upon increasing the glutamate concentration. However, from a concentration of 0.4 mM onwards, any increase in glutamate concentration causes a rise in potential as expected

FIG. 4(G)-(I) provide insight into the effect of scaling the electrode dimension on the sensing performance. Specifically, these figures highlight the sensitivity difference by systematically varying the anode and cathode size, respectively, while keeping the size of the other electrode constant at 2.5 mm in diameter. Controlling the amount of enzyme solution/Pt—C and Nafion mixtures added to the electrodes ensures the same density of surface functional components in all groups. Decreasing the surface area of the electrode results in an increase in electrode resistance. For example, when the surface area decreases from 0.442 mm² to 0.096 mm², the impedance increases from approximately 4.6×10⁴ to 3×10⁶Ω (please note that the surface area of the opening for the 1000 micron electrode is 0.244 mm², and that of the 750 micron electrode is 0.442 mm² due to a small amount of UV gel flowing into the 1000 micron aperture, thereby decreasing the biochemical interface area). To avoid the voltage division effect across the solution-sensor interface caused by large increases in impedance, replacing the 100 kΩ resistor with a 10 MΩ alternative can be helpful in more efficiently collecting voltage signals for miniaturized electrodes. Additionally, implementing a differential amplifier with a 10× gain across the load resistor further amplifies the signal and maximizes the signal to noise ratio (FIG. 4(G)). The progressive increase in impedance with decreasing effective surface area for both the anode and cathode explains the phenomenon of decreasing sensitivity (FIG. 4(H)-(I)) due to restricted current flow. Specifically, the sensitivity decreases from 186.72 to 66.64 mV mM⁻¹ the anode diameter decreases from 1 to 0.35 mm, and the value decreases from 231.11 to 80.1 mV mM⁻¹ when the cathode diameter decreases from 1 mm to 0.35 mm.

We also evaluated the importance of aeration for optimal cathodic performance. Since protons and oxygen are reduced to water at the cathode, insufficient aeration can result in a limited sensor response, particularly at lower glutamate concentrations. This is demonstrated by the differences in sensing performance without and with aeration, where the former is not able to quantify glutamate at concentrations between 0.2 and 0.4 mM.

We also evaluated the performance of a thin film electrode when both the diameter of the anode and cathode are miniaturized to 500 μm. The miniaturized device displays a sensitivity of 185.98 m V mM⁻¹ and can detect glutamate at low concentrations of 0.015 mM.

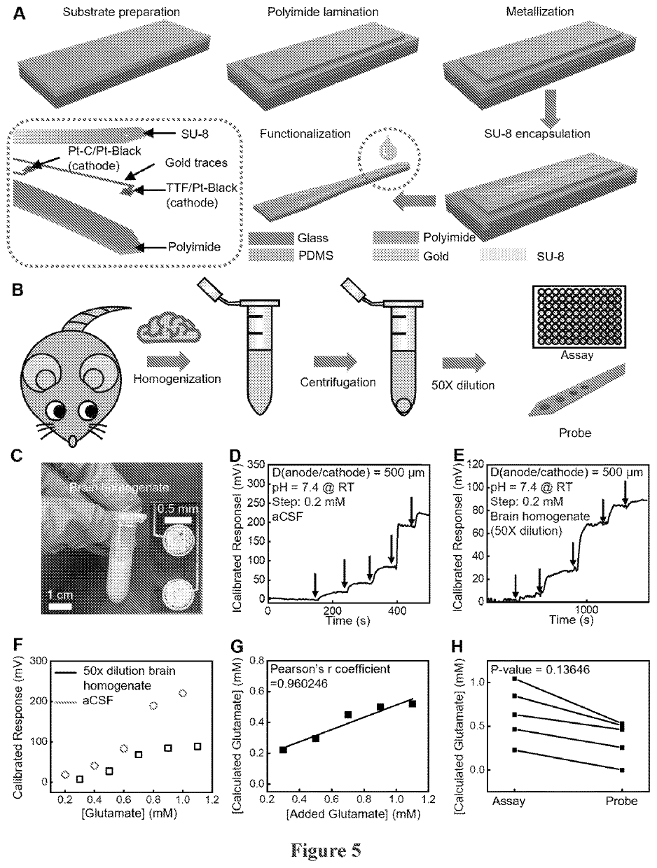

Testing of performance of microfabricated probes in mouse brain homogenate. The knowledge and understanding acquired in the preceding sections have informed the design and development of sensing probes on polymeric substrates using standard microfabrication techniques in a high throughput manner (FIG. 5(A)). Briefly, the fabrication of the glutamate probes involves laminating a flexible Kapton film on a glass slide with uncured polydimethylsiloxane (PDMS) and heating to form the substrate. Subsequent deposition, lithographic patterning, and etching yield electrodes thin-film Cr/Au electrodes as the cathode and anode. Functionalizing the electrodes based on the protocols described earlier completes the fabrication process of the glutamate probes. Further details about the fabrication process are included below.

Characterizing the performance of the biofuel cell probes in brain tissue ex vivo assesses their capability of operating in a complex biological environment. Homogenizing a mouse brain in 1 mL aCSF followed by centrifuging the sample (10,000 RPM for 10 minutes) yields supernatants providing a tissue environment. The use of a commercial assay kit (MAK004, Sigma Aldrich) to test brain homogenates (diluted 5-fold) indicates a glutamate concentration of 1.018 mM. As the concentration of glutamate in the brain homogenate falls outside the detection limit of the biofuel cell, a 50× dilution of the homogenate to a concentration of 0.1 mM is necessary to establish the baseline calibration. Adding concentrated glutamate (1 M) into the base solution forms a series of test solutions for the characterization of the probes. Comparing the accuracy for determining the glutamate concentration in the diluted brain homogenate with the commercial glutamate assay evaluates the performance of the microfabricated probes (FIG. 5(B)). FIG. 5(C) showcases the brain homogenate sample following centrifugation while the image on the bottom right corner is a microscope image of the probe (electrode diameter: 500 μm). FIGS. 5(D) and 5(E) show the calibration plots for a pair of 500-μm diameter electrodes obtained in 1× aCSF and brain homogenate (50× dilution), respectively. The extracted sensitivity is 272 and 109 mV mM⁻¹, as summarized in FIG. 5(F). The discrepancy might be due to the presence of interferents in the complex biological environment. FIG. 5(G) shows the Pearson's coefficient (r=0.960246) between the added glutamate concentration and the calculated glutamate concentration based on the calibration curve of the probe in aCSF. A similar experimental design was also used but glutamate concentration was measured using commercial assay kit. FIG. 5(H) presents the comparison between the glutamate concentration measured using the probe and the glutamate assay kit in a 50× brain homogenate dilution, yielding a p value of 0.136. The slight discrepancy observed may be attributed to the surface saturation issue of the sensor at the biochemical interface, whereas the commercial assay allows for uniform spreading of analytes in the wells. Nevertheless, the results of the validation experiments in brain homogenate suggest that the probes inspired by biofuel cells have the potential to quantitatively measure glutamate in brain tissues during future in vivo studies.

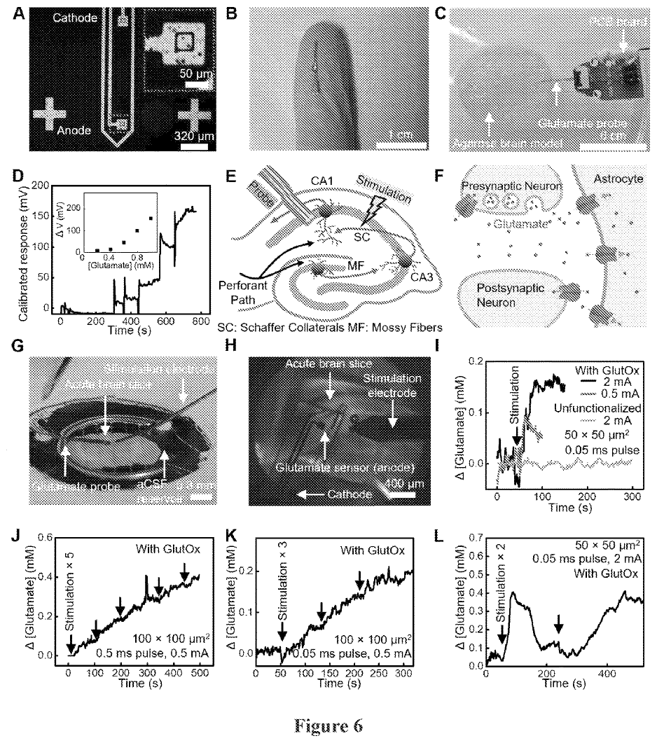

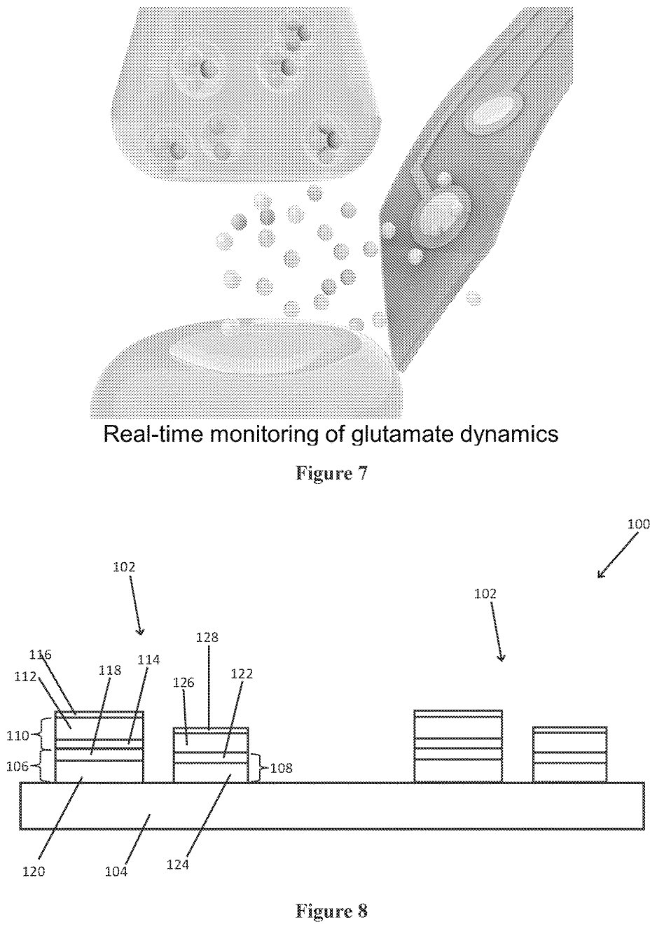

Ex vivo studies of dynamic glutamate release from the hippocampal circuit in mice. Building on the successful development of glutamate sensors, the study leverages these probes to quantitatively analyze the release of glutamate from acute mouse brain slices. Further scaling down the form factors of the probes to nearly cellular-scale dimensions (as small as 50 by 50 μm² for the sensing area) enhances the spatial resolution and biocompatibility for future in vivo applications. This will enable real-time visualization of dynamic changes in glutamate concentration at the tissue-device interface. FIGS. 6(A) and 6(B) show a microscopic image and photograph of a representative miniaturized device, respectively, providing a visual representation of the size and design parameters of the probes. FIG. 6(C) shows a mechanical test of the probe using a brain model made of a 0.6 w/w % agarose solution in DI water, which has a similar young's modulus to a mouse brain. The photograph demonstrates that the probe can penetrate the agarose model with minimal damage to the biochemical interface and gold traces.

FIG. 6(D) presents the calibration curve for the 50 by 50 μm² and 100 by 100 μm² glutamate probe, respectively, created by using a modified functionalization protocol: The procedure starts with adding 0.05 μL of 20 mg mL⁻¹ TTF to the anode and 0.05 μL of the Pt—C/Nafion mixture to the cathode. Drop-casting 0.5 μL of GlutOx at a concentration of 100 U ml⁻¹ periodically to the surface of the anode minimizes overflowing of the enzyme solution from the electrode surface. The 50 by 50 μm² glutamate probe exhibits good linearity over the testing range (R²>0.92) and a sensitivity of 167.13 mV mM⁻¹ using a 250 MΩ resistor and 10× amplification.

The study utilizes the hippocampal circuit in mice to evaluate the performance of the miniaturized probes in detecting synaptically released glutamate, as shown in FIG. 6(E): when Schaffer collateral fibers originating from CA3 neurons are stimulated electrically using a bipolar electrode, glutamatergic synaptic vesicles undergo exocytosis in the CA1 Stratum Radiatum region (FIG. 6(F)). Subsequently, glutamate is released from the presynaptic neuron into the synaptic cleft via exocytosis. Excitatory amino acid transporters (EAAT) will then facilitate the uptake of glutamate and transport glutamate from the synaptic cleft back into the neurons and astrocytes, thereby reducing the overall glutamate concentration in the extracellular space and preventing the continuous firing of action potentials (indicated by blue arrows). FIGS. 6(G) and (H) depict the experimental setup, which includes a stimulation electrode and a glutamate probe laminated onto the surface of the CA1 region of the hippocampus. FIG. 6(I) shows changes in local glutamate concentration obtained using the probe with and without key functional layers on the anode (i.e., TTF and GlutOx). All stimulations use a bi-phasic pulse width of 0.05 ms. The results suggest an immediate increase in the recorded signal following the stimulation, as detected using the functionalized probe, whereas the signal captured by the unfunctionalized probe remains constant due to the lack of bio-recognition elements for glutamate. The increased voltage indicates the successfully detection of the synaptic release event using the glutamate probe following the hippocampal circuit model depicted in FIGS. 6(E) and (F). For the same functionalized probe, increasing the intensity of the stimulation current (from 0.5 to 2 mA) results in an increased amplitude in detected glutamate concentration, possibly due to more axons being recruited by larger stimulus intensity, resulting in more synaptic release. It is important to note that the observed changes in glutamate concentration (approximately 0.05 mM to 0.15 mM) are lower than the reported values in the extracellular space according to the literature. One possible explanation for this observation could be that, in this study, the probe is laminated onto the surface of the brain slice rather than penetrating through the tissue. This may have caused any glutamate to diffuse across the surface of the brain tissue and into the surrounding aCSF reservoir, resulting in a lower glutamate concentration and a longer response time.

The data in FIG. 6(J) illustrates the progressive rise in glutamate concentration, as measured by a 100 by 100 μm² probe, during five consecutive stimulations (as indicated by the arrows) with a pulse width of 0.5 ms and a current intensity of 0.5 mA. Each pulse results in the release of a similar amount of glutamate in the local area, creating a stepwise pattern. FIG. 6(K) depicts the outcomes obtained using the same 100 by 100 μm² probe from a comparable 0.5 mA pulse stimulation, but with a stimulation period of 0.05 ms. The results indicate that in this experimental setup, increasing the pulse width does not have a significant impact on the amount of glutamate released from each stimulation. With a single pulse (width: 0.05 ms, intensity: 2 mA) applied, and no further stimulations over a period of approximately 300 s, the concentration of glutamate gradually decreases and returns to the baseline (as depicted in FIG. 6(L)). This observation is likely due to a combined effect of glutamate uptake by transporters in the extracellular space, as well as diffusion in the solution. Following the application of another pulse, the signal increases once more and reaches a similar amplitude as expected. Overall, the results obtained indicate the tremendous potential of the miniaturized probes in the quantitative analysis of glutamate dynamics with a spatial precision. This could lead to a better understanding of previously unknown molecular and cellular mechanisms underlying the structural and functional reorganization of neural circuits

### CONCLUSION

In summary, this study illustrates the feasibility of utilizing a biofuel cell design in a flexible and miniaturized sensing probe to enable the continuous, real-time detection of glutamate in complex biological environments. The findings indicate that the biofuel cell configuration exhibits sensitivity to glutamate, with detectable concentrations spanning from about 0.05 mM to 1.0 mM. This research comprises comprehensive investigations of critical parameters that influence the sensing performance, as well as the necessary requirements and considerations for constructing miniaturized sensing probes. Testing the resulting sensing platform with biological samples, including 50× dilutions of mouse brain homogenates and mouse brain slices, validates the effectiveness of the system. The results obtained from experiments utilizing the hippocampal circuit in mice suggest that the miniaturized probes can detect the dynamics of synaptically released glutamate triggered by electrical stimulation. Future studies will aim to explore changes in glutamate concentrations in different regions of the brain, such as the cortex or the ventral tegmental area (VTA), by employing miniaturized probes.

Additional efforts will focus on further reducing the overall size of the electrode for improved spatial resolution, enhancing the sensitivity, and minimizing the overall variation in sensitivity between devices. Furthermore, incorporating other wireless components, such as a Bluetooth or near-field communication (NFC) chip, could improve the device's implantability and enable potential in vivo testing. This device could have potential applications in glutamate concentration mapping with high spatial and temporal resolution. A multiplexed system that incorporates an array of miniaturized anodes connected to a common cathode could enable the mapping of glutamate concentration across various brain regions simultaneously. Disease state models of TBI could be utilized to investigate the extent to which glutamate concentration increases in distinct brain regions and their correlation with elevated neuronal firing activity. Therefore, a multiplexed device could have diagnostic potential for detecting the onset and progression of brain injury by enabling the real-time monitoring of glutamate concentrations across multiple brain regions. Additionally, to enhance the diagnostic accuracy of the device, it would be valuable to increase the sensor's detection speed, enabling it to recognize rising levels of glutamate concentration within a few milliseconds. This improvement would allow the device to detect increases in glutamate concentration resulting from each individual action potential, making it a useful tool for mapping out glutamate cell signaling pathways in both healthy and diseased states. This technology could be particularly beneficial to healthcare professionals by aiding in the early detection of diseases like stroke, epilepsy, and schizophrenia that can be caused by elevated levels of glutamate.

The sensors, devices, and methods of the appended claims are not limited in scope by the specific sensors, devices, and methods described herein, which are intended as illustrations of a few aspects of the claims. Any sensors, devices, and methods that are functionally equivalent are intended to fall within the scope of the claims. Various modifications of the sensors, devices, and methods in addition to those shown and described herein are intended to fall within the scope of the appended claims. Further, while only certain representative sensors, devices, and methods steps disclosed herein are specifically described, other combinations of the sensors, devices, and methods also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated.

The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than where noted, all numbers expressing geometries, dimensions, and so forth used in the specification and claims are to be understood at the very least, and not as an attempt to limit the application of the doctrine of equivalents to the scope of the claims, to be construed in light of the number of significant digits and ordinary rounding approaches.

1. A biosensor for detecting an analyte of interest, the biosensor comprising one or more electrochemical sensors disposed on a flexible substrate,
wherein each of the one or more electrochemical sensors comprise:
a first electrode disposed on a flexible substrate;
a second electrode disposed on the flexible substrate and in electrochemical contact with the first electrode; and
an active layer comprising an enzyme disposed on the first electrode;
wherein the analyte of interest is a biochemical substrate for the analyte of interest.

2. The biosensor of claim 1, wherein the active layer further comprises an electron mediator, such as tetrathiafulvalene.

3. The biosensor of claim 1, further comprising a polymer network disposed on the active layer.

4. The biosensor of claim 3, wherein the polymer network is permeable towards the analyte of interest.

5. The biosensor of claim 3, wherein the polymer network comprises a sulfonated tetrafluoroethylene based fluoropolymer-copolymer (e.g., Nafion).

6. The biosensor of claim 3, wherein the polymer network comprises a polymer membrane.

7. The biosensor of claim 6, wherein the membrane exhibits a 1100 g/mol equivalent weight and a thickness of 0.007 inches.

8. The biosensor of claim 3, wherein the polymer network is substantially impermeable to an interferent.

9. The biosensor of claim 3, wherein the polymer network is configured to immobilize the active layer on the first electrode.

10. The biosensor of claim 3, wherein the polymer network is BSA surface-modified.

11. The biosensor of claim 1, wherein the first electrode comprises a high surface area conductive material, such as Pt black, disposed on a conductive material, such as a metal (e.g., Au).

12. (canceled)

13. The biosensor of claim 1, further comprising a third electrode disposed on the flexible substrate and in electrochemical contact with the first electrode and the second electrode.

14. The biosensor of claim 1, wherein the enzyme is chosen from glucose oxidase, glucose dehydrogenase, NADH oxidase, uricase, urease, creatininase, sarcosine oxidase, creatinase, creatine kinase, creatine amidohydrolase, cholesterol esterase, cholesterol oxidase, glycerol kinase, hexokinase, glycerol-3-phosphate oxidase, lactate oxidase, lactate dehydrogenase, alkaline phosphatase, alanine transaminase, aspartate transaminase, amylase, lipase, esterase, gamma-glutamyl transpeptidase, a glutamate oxidase (e.g., L-glutamate oxidase), pyruvate oxidase, diaphorase, bilirubin oxidase, and mixtures thereof.

15. (canceled)

16. The biosensor of claim 1, wherein the biosensor further comprises circuitry configured to measure a current flowing between the first electrode and the second electrode and a processor configured to calculate a concentration of the analyte of interest in contact with the first electrode based on the current flowing between the first electrode and the second electrode.

17. (canceled)

18. The biosensor of claim 1, wherein the biosensor further comprises circuitry configured to measure a potential between the first electrode and the second electrode and a processor configured to calculate a concentration of the analyte of interest in contact with the first electrode based on the potential between the first electrode and the second electrode.

19. (canceled)

20. The biosensor of claim 1, wherein the biosensor comprises a plurality of the electrochemical sensors disposed on the flexible substrate in a 2-dimensional or 3-dimensional array.

21. (canceled)

22. The biosensor of claim 20, wherein the biosensor further comprises circuitry configured to measure a current flowing between the first electrode and the second electrode in each of the plurality of the electrochemical sensors and a processor configured to calculate a concentration of the analyte of interest in contact with the first electrode in each of the plurality of the electrochemical sensors based on the current flowing between the first electrode and the second electrode;
wherein the processor is further configured to correlate the concentration of the analyte of interest in contact with the first electrode of each of the plurality of the electrochemical sensors with a 2-dimensional or 3-dimensional location of each of the plurality of the electrochemical sensors within the 2-dimensional or 3-dimensional array, thereby providing 2-dimensional or 3-dimensional information regarding the concentration of the analyte of interest in proximity to the biosensor.

23. (canceled)

24. (canceled)

25. The biosensor of claim 20, wherein the biosensor further comprises circuitry configured to measure a potential between the first electrode and the second electrode in each of the plurality of the electrochemical sensors and a processor configured to calculate a concentration of the analyte of interest in contact with the first electrode in each of the plurality of the electrochemical sensors based on the potential between the first electrode and the second electrode;
wherein the processor is further configured to correlate the concentration of the analyte of interest in contact with the first electrode of each of the plurality of the electrochemical sensors with a 2-dimensional or 3-dimensional location of each of the plurality of the electrochemical sensors within the 2-dimensional or 3-dimensional array, thereby providing 2-dimensional or 3-dimensional information regarding the concentration of the analyte of interest in proximity to the biosensor.

26. (canceled)

27. (canceled)

28. The biosensor of claim 1, wherein the biosensor is battery-free and is not electrically connected to a power source.

29. (canceled)

30. (canceled)

31. (canceled)

32. A method of detecting an analyte of interest in a medium comprising contacting the medium with the biosensor of claim 1.

33. (canceled)

34. (canceled)
